(19) United States
(12) Patent Application Publication
(10) Pub. No.: US 2004/0121014 A1
(43) Pub. Date: Jun. 24, 2004

(54) Method for treating and/or preventing retinal diseases with sustained release corticosteroids

(75) Inventors: Hong Guo, Belmont, MA (US); Paul Ashton, Boston, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/714,677

(22) Filed: Nov. 14, 2003

Related U.S. Application Data
(63) Continuation-in-part of application No. 10/253,825, filed on Sep. 25, 2002.
Application No. 10/253,825 is a continuation of application No. 09/735,636, filed on Dec. 14, 2000, now Pat. No. 6,548,078.
Application No. 09/735,636 is a continuation of application No. 09/273,548, filed on Mar. 22, 1999, now Pat. No. 6,217,895.

(51) Int. Cl. A61K 31/573; A61K 9/24

(52) U.S. Cl. 424/471; 514/179

(57) ABSTRACT

The present invention relates to a method for administering a corticosteroid to a posterior segment of an eye. In the method, a sustained release device is implanted to deliver the corticosteroid to the eye. The aqueous corticosteroid concentration remains less than vitreous corticosteroid concentration during release of the corticosteroid from the device.

### RELATED APPLICATIONS

This application is a continuation-in-part of U.S. application Ser. No. 10/253,825 filed Sep. 25, 2002, which is a continuation of U.S. application Ser. No. 09/735,636 filed Dec. 14, 2000, now U.S. Pat. No. 6,548,078, which is a continuation of U.S. application Ser. No. 09/273,548 filed Mar. 22, 1999, now U.S. Pat. No. 6,217,895, entitled “Method for Treating and/or Preventing Retinal Diseases with Sustained Release Corticosteroids”, all of which are incorporated by reference herein in their entirety.

### FIELD OF THE INVENTION

The present invention relates to the field of controlled pharmaceutical delivery, particularly to corticosteroids.

### BACKGROUND OF THE INVENTION

Compounds classified as corticosteroids, such as triamcinolone, can effectively treat some forms of neovascularization such as corneal neovasularization. In general, corticosteroids have been unsuccessful in treating neovascularization of the posterior segment. In many patients, these compounds cause undesirable side effects. These adverse affects include elevations in intraocular pressure and the formation of, or acceleration of, the development of cataracts. Elevations in intraocular pressure are of particular concern in patients who are already suffering from elevated intraocular pressure, such as glaucoma patients. Moreover, a risk exists that the use of corticosteroids in patients with normal intraocular pressure will cause elevations in pressure that result in damage to ocular tissue. Since therapy with corticosteroids is frequently long term, i.e., several days or more, a potential exists for significant damage to ocular tissue as a result of prolonged elevations in intraocular pressure attributable to that therapy.

One approach to solving the foregoing problems has been to search for specific compounds which are effective in treating neovascularization without elevating intraocular pressure. Another approach has been to administer corticosteroids in conjunction with another drug to “block” or reduce the IOP elevating effects of the corticosteroids or to treat IOP elevation separately with another drug. A further approach has been to intravitreally inject corticosteroids to treat ocular neovascularization.

There exists a need for an improved method for treating and/or preventing retinal diseases with corticosteroids.

### DISCLOSURE OF THE INVENTION

An object of the present invention is to provide a method for treating and/or preventing ocular diseases which have neovascularization as a component with corticosteroids without the associated adverse side effects.

Additional objects, advantages and other features of the invention will be set forth in the description which follows and in part will become apparent to those having ordinary skill in the art upon examination of the following or may be learned from the practice of the invention. The objects and advantages of the invention may be realized and obtained as particularly pointed out in the appended claims.

According to the present invention, the foregoing and other objects are achieved in part by a method for administering a corticosteroid to a posterior segment of an eye, the method comprising the step of:

implanting a sustained release device to deliver the corticosteroid to the vitreous of the eye wherein aqueous corticosteroid concentration is less than vitreous corticosteroid concentration during release.

In accordance with the present invention, the foregoing and other advantages are also achieved in part by an implantable, sustained release device for administering a corticosteroid to a posterior segment of an eye, the device comprising:

a corticosteroid, wherein the device is configured to provide sustained release of the corticosteroid to the vitreous of the eye such that aqueous corticosteroid concentration remains less than vitreous corticosteroid concentration during the release.

Additional objects and advantages of the present invention will become readily apparent to those skilled in this art from the following detailed description, wherein embodiments of the invention are described simply by way of illustrating of the best mode contemplated in carrying out the invention. As will be realized, the invention is capable of other and different embodiments, and its several details are capable of modifications in various obvious respects, all without departing from the invention. Accordingly, the drawings and description are to be regarded as illustrative in nature and not as restrictive.

### BRIEF DESCRIPTION OF DRAWINGS

FIG. 1 is an enlarged view of one embodiment of the sustained release drug delivery device showing inner core, first coating layer and second coating layer.

FIG. 2 is an enlarged cross sectional schematic of one embodiment of the sustained release drug delivery device showing inner core, first coating layer and second coating layer.

FIG. 3 is an enlarged view of an embodiment of the sustained release drug delivery device showing inner drug core and permeable polymer coating.

FIG. 4 is an enlarged view of an embodiment of the sustained release drug delivery device showing inner drug core, permeable polymer coating of the present invention and an extension of the polymer coating layer as a means for attachment.

FIG. 5 is an enlarged view of an embodiment of the sustained release drug delivery device showing inner drug core, permeable polymer coating layer and an extension of the polymer coating layer containing a support ring as a means for attachment wherein the support ring allows enough space for a suture to be passed between the drug core and the support ring.

FIG. 6 is an enlarged view of an embodiment of the sustained release drug delivery device showing inner drug core, permeable polymer coating layer and an extension of the polymer coating layer containing a support ring as a means for attachment wherein the support ring forms a loop through which a suture can be passed.

FIG. 7 is an enlarged view of an embodiment of the sustained release drug delivery device showing inner drug core, permeable polymer coating layer and an extension of the polymer coating layer containing a backing material as a means for attachment.

FIG. 8 is an enlarged view of an embodiment of the sustained release drug delivery device showing inner drug core, permeable polymer coating layer and an extension of the polymer coating layer containing a backing material as a means for attachment.

FIG. 9 is an enlarged view of one embodiment of the sustained release drug delivery device showing inner core, first coating layer, second coating layer and third coating layer.

FIG. 10A is an enlarged view of the impermeable polymer. FIG. 10B is an enlarged view of the second coating layer including the impermeable film and impermeable disc.

FIG. 11 shows the sustained release profile of a 2 mg fluocinolone acetonide implant in buffer over 100 days. The mean release rate was 2.1+/−0.26 μg/day.

FIG. 12 shows the vitreous and aqueous levels of fluocinolone acetonide after implantation of a sustained release device. Animals were sacrificed at 4 weeks, 20 weeks, and 1 year. FIG. 12 shows that therapeutic levels are maintained in the vitreous while drug levels in the aqueous humor were below the detection limit of the assay.

### DESCRIPTION OF THE INVENTION

The present invention provides a method for delivering a therapeutic amount of a corticosteroid to the vitreous of an eye but prevents toxic amounts of the corticosteroid from accumulating in the aqueous. The method comprises the step of implanting a sustained release device comprising a corticosteroid to the posterior segment to deliver the corticosteroid to the vitreous wherein aqueous corticosteroid concentration is less than vitreous corticosteroid concentration during release of the corticosteroid.

The present invention is particularly effective in treating diseases of the retina, retinal pigment epithelium (RPE) and choroid. These diseases include, for example, ocular neovascularization, ocular inflammation and retinal degenerations. Specific examples of these disease states include diabetic retinopathy, chronic glaucoma, retinal detachment, sickle cell retinopathy, senile macular degeneration, retinal neovascularization, subretinal neovascularization; rubeosis iritis inflammatory diseases, chronic posterior and pan uveitis, neoplasms, retinoblastoma, pseudoglioma, neovascular glaucoma; neovascularization resulting following a combined vitrectomy and lensectomy, vascular diseases retinal ischemia, choroidal vascular insufficiency, choroidal thrombosis, neovascularization of the optic nerve, diabetic macular edema, cystoid macular edema, macular edema, retinitis pigmentosa, retinal vein occlusion, proliferative vitreoretinopathy, angioid streak, and retinal artery occlusion, and, neovascularization due to penetration of the eye or ocular injury.

Examples of corticosteroids useful in the present invention include, for example, triamcinolone, dexamethasone, fluocinolone, cortisone, prednisolone, flumetholone, and derivatives thereof.

By “sustained release device” it is meant a device that releases drug over an extended period of time in a controlled fashion. Examples of sustained release devices useful in the present invention may be found in, for example, U.S. Pat. No. 5,378,475 and U.S. Pat. No. 5,773,019, and U.S. Ser. No. 08/919,221 filed on Aug. 28, 1997, now U.S. Pat. No. 5,902,598.

For example, U.S. Pat. No. 5,378,475 (the “'475 patent”) teaches a device includes an inner core or reservoir which contains an agent effective in obtaining a desired effect. The device further includes a first coating layer and a second coating layer. The first coating layer covers only a portion of the inner core and is impermeable to the passage of the agent. The second coating layer covers all of the inner core and the first coating layer and is permeable to the passage of the agent. The portion of the inner core that is not coated with the first coating layer facilitates passage of the agent through the second coating layer.

Specifically, the first coating layer is positioned between the inner core and the second coating layer such that it blocks the passage of the agent through the adjacent portions of the second coating layer thus controlling the rate of passage of the agent.

FIG. 1 illustrates one embodiment of the sustained release drug delivery device of the present invention. While the device shown in FIG. 1 is cylindrical, the device could be any shape. The device comprises an inner core or reservoir 5, an impermeable coating 10 which is impermeable to the passage of the agent in the core or reservoir 5, and a permeable coating 15 which is permeable to the passage of the agent in the core or reservoir 5. FIG. 1 further shows an impermeable cap 20 and suture tag 25.

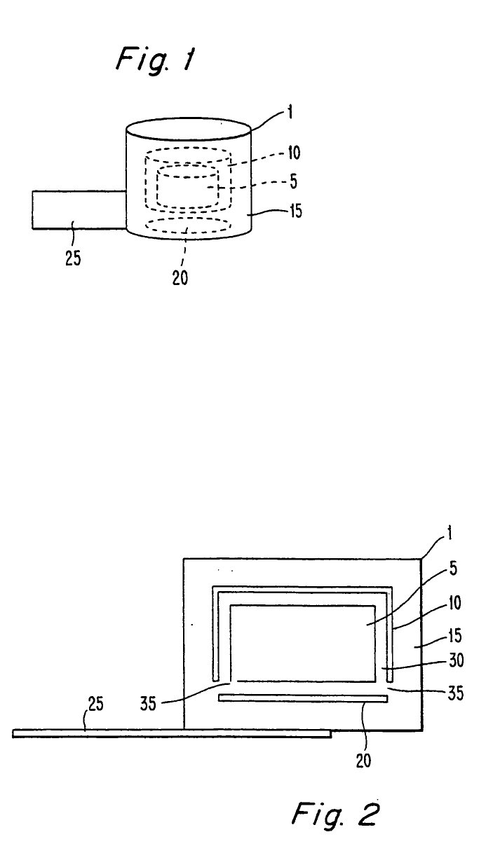

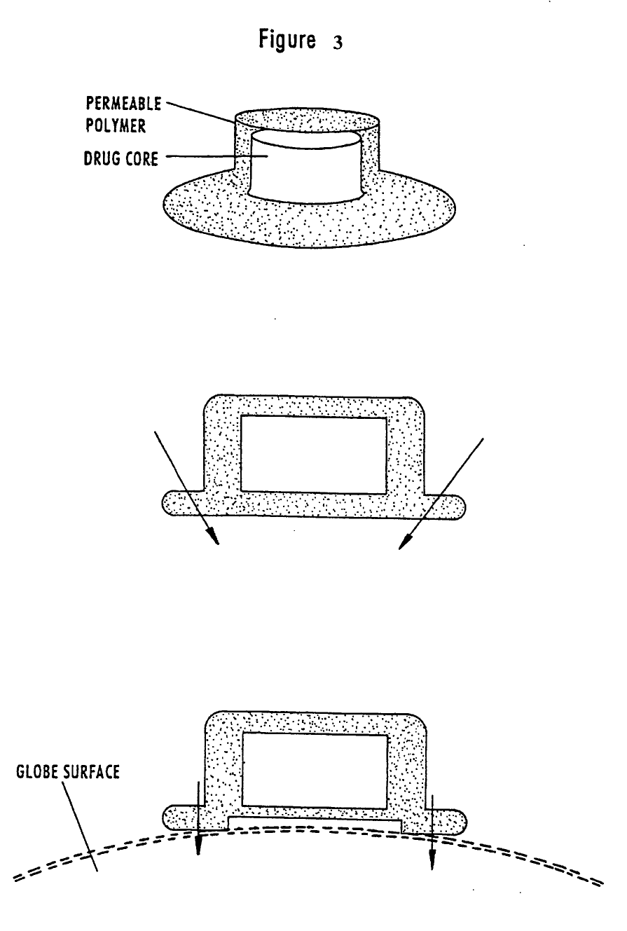

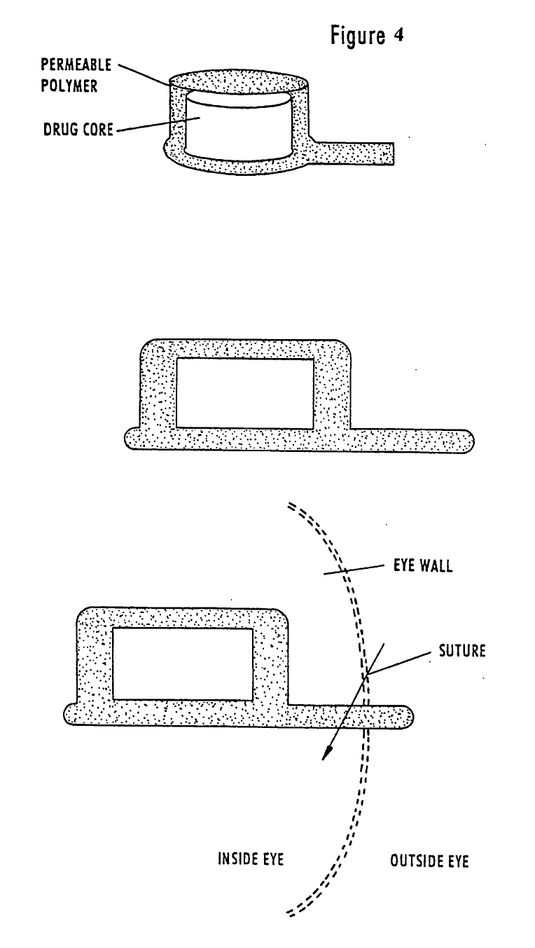

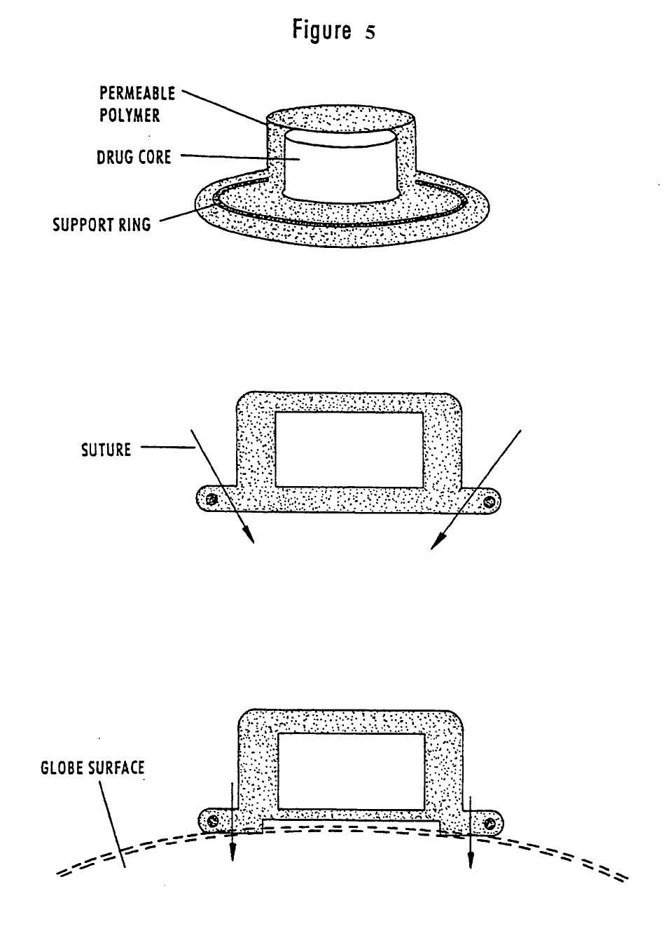

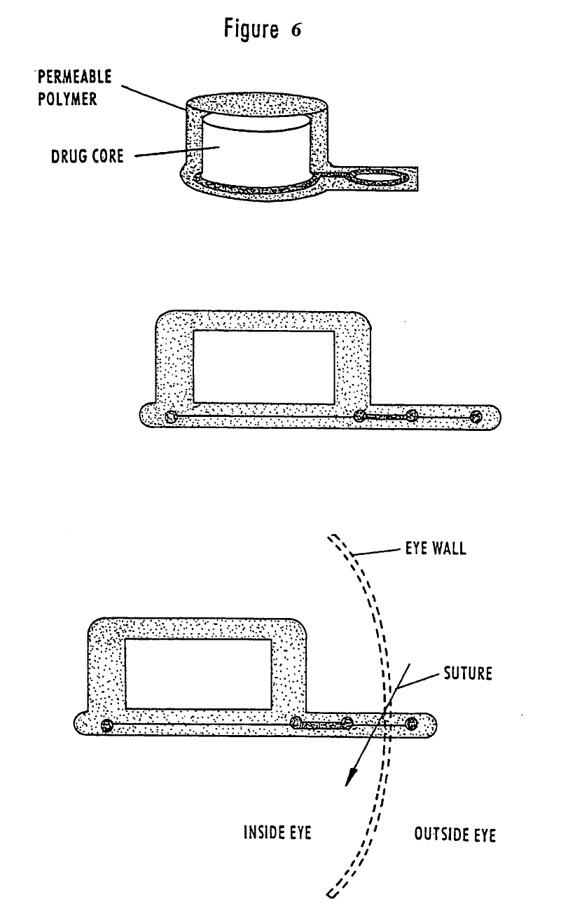

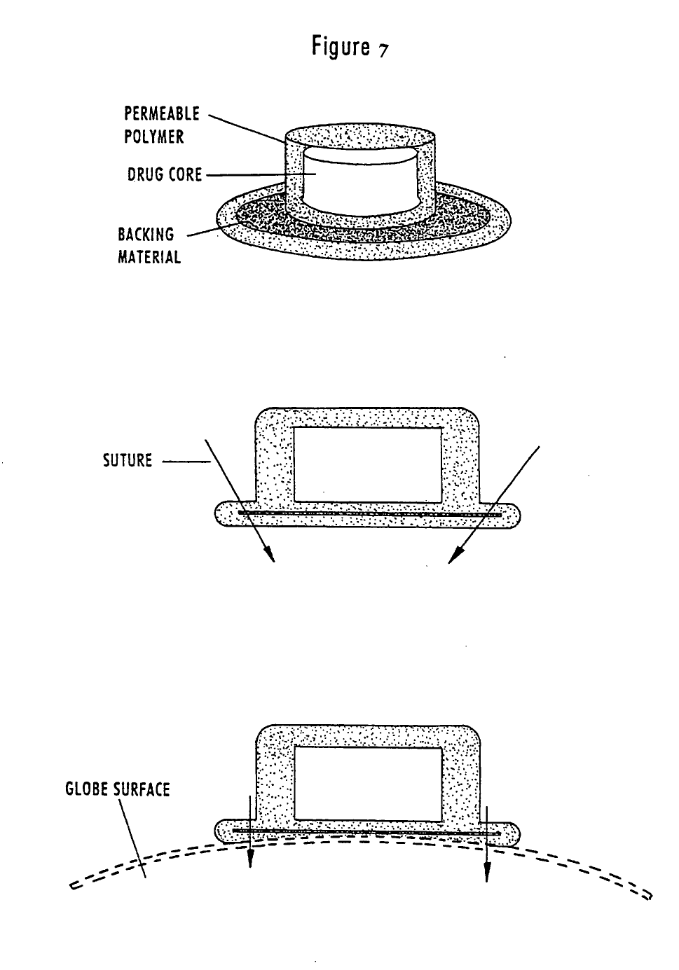

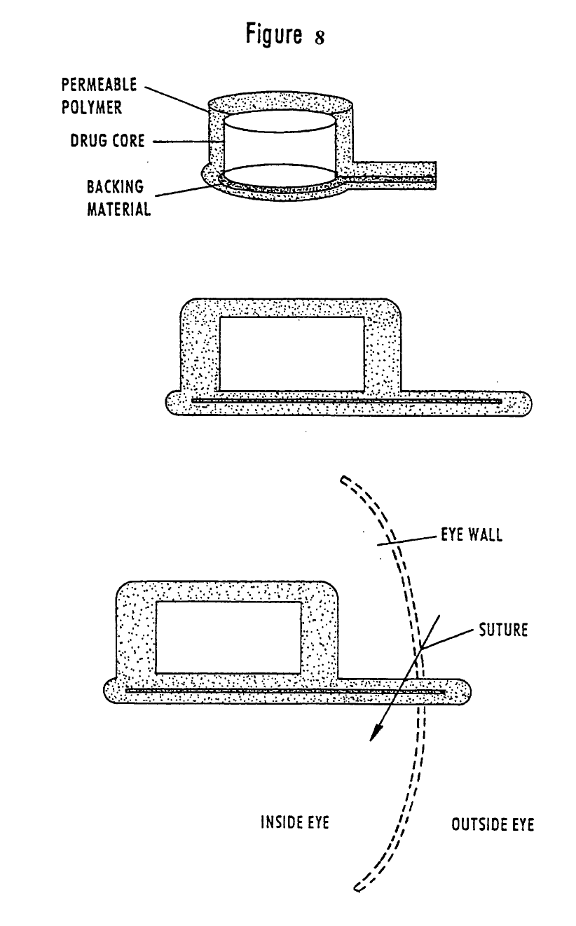

FIG. 2 illustrates, in cross section, the device shown in FIG. 1. As illustrated, there may be a permeable coating 30 between the core or reservoir 5 and the impermeable coating 10. The permeable coating 30 may be made of the same material as the permeable coating 15. In the embodiment illustrated in FIG. 2, the impermeable cap 20 is positioned such that there is a passage 35 which allows passage of the agent in the core or reservoir. The impermeable coating 20 is positioned between the permeable coating 15 and the reservoir or core 5. The suture tag 25 is attached to the permeable coating 15.

The devices are particularly suitable for treating ocular conditions such as glaucoma, proliferative vitreoretinopathy, diabetic retinopathy, uveitis, and keratitis. The devices are also particularly suitable for use as an ocular device in treating mammalian organisms suffering from cytomegalovirus retinitis wherein the device is surgically implanted within the vitreous of the eye.

The first layer must be selected to be impermeable, as described above, to the passage of the agent from the inner core out to adjacent portions of the second coating layer. The purpose is to block the passage of the agent to those portions and thus control the release of the agent out of the drug delivery device.

The composition of the first layer, e.g., the polymer, must be selected so as to allow the above-described controlled release. The preferred composition of the first layer will vary depending on such factors as the active agent, the desired rate of control and the mode of administration. The identity of the active agent is important since the size of the molecule, for instance, is critical in determining the rate of release of the agent into the second layer.

Since the first coating layer is essentially impermeable to the passage of the effective agent, only a portion of the inner core or reservoir may be coated with the first coating layer. Depending on the desired delivery rate of the device the first coating layer may coat only a small portion of the surface area of the inner core for faster release rates of the effective agent or may coat large portions of the surface area of the inner core for slower release rates of the effective agent.

For faster release rates, the first coating layer may coat up to 10% of the surface area of the inner core. Preferably, approximately 5-10% of the surface area of the inner core is coated with the first coating layer for faster release rates.

For slower release rates, the first coating layer may coat at least 10% of the surface area of the inner core. Preferably, at least 25% of the surface area of the inner core is coated with the first coating layer. For even slower release rates, at least 50% of the surface area may be coated. For even slower release rates, at least 75% of the surface area may be coated. For even slower release rates, at least 95% of the surface area may be coated.

Thus, any portion of the surface area of the inner core up to but not including 100% may be coated with the first coating layer as long as the desired rate of release of the agent is obtained.

The first coating may be positioned anywhere on the inner core, including but not limited to the top, bottom or any side of the inner core. In addition, it could be on the top and a side, or the bottom and a side, or the top and the bottom, or on opposite sides or on any combination of the top, bottom or sides.

The second layer of the device of the present invention must be biologically compatible with body fluids and eye tissues, essentially insoluble in body fluids which the material will come in contact and permeable to the passage of the agent or composition effective in obtaining the desired effect.

The effective agent diffuses in the direction of lower chemical potential, i.e., toward the exterior surface of the device. At the exterior surface of the device, equilibrium is again established. When the conditions on both sides of the second coating layer are maintained constant, a steady state flux of the effective agent will be established in accordance with Fick's Law of Diffusion. The rate of passage of the drug through the material by diffusion is generally dependent on the solubility of the drug therein, as well as on the thickness of the wall. This means that selection of appropriate materials for fabricating the wall will be dependent on the particular drug to be used.

When such devices are prepared for implantation within the vitreous of the eye, it is preferred that the device does not exceed about 5 millimeters in any direction. Thus, the cylindrical device shown in FIG. 2 would preferably not exceed 5 millimeters in height or diameter. In addition, the preferred thickness of the first coating layer ranges from about 0.1 to about 1.0 millimeters. The preferred thickness of the second coating layer ranges from about 0.1 to about 2.0 millimeters.

U.S. Pat. No. 5,773,019 (the “'019 patent”) describes a device including an inner core comprising an effective amount of a low solubility agent, and a non-bioerodible polymer coating layer, the polymer layer permeable to the low solubility agent, wherein the polymer coating layer covers the inner core.

Once implanted, the device gives a continuous supply of the agent to internal regions of the body without requiring additional invasive penetrations into these regions. Instead, the device remains in the body and serves as a continuous source of the agent to the affected area. In another embodiment, the device further comprises a means for attachment, such as an extension of the non-erodible polymer coating layer, a backing member, or a support ring. In a preferred embodiment, the device is suitable for direct implantation into the vitreous of the eye.

The device according to the present invention permits prolonged constant release of low solubility agents over a specific period of months (e.g., 3 months, 6 months) or years (e.g., 1 year, 5 years, 10 years, 20 years) until the agent is substantially used up.

This device enables a large variety of drugs and other agents to be delivered into any internal region of the body, preferably the eye. Cyclosporine A in low solubility form is a preferred drug used in the delivery device.

The non-bioerodible polymer coating layer of the present invention may completely or partially cover the inner core. In this regard, any portion of the surface area of the inner core up to and including 100% may be coated with the polymer coating layer as long as the pellet is protected against disintegration, prevented from being physically displaced from its required site, and as long as the polymer coating layer does not adversely retard the release rate.

The drug delivery device of the present invention is particularly suitable for direct surgical implantation into the eye.

The entire structure is made of material which is compatible with the human tissue with which it comes in contact. In a preferred embodiment the material of the device is polyvinyl alcohol. If a backing member is present in a preferred embodiment, the backing member may be composed of any material tolerated by the human body, preferably ethylene vinyl acetate, Teflon, silicone, silastic and nylon.

U.S. Pat. No. 5,902,598 (the “'598 patent”) further teaches a device, in one embodiment, including an inner core or reservoir which contains an agent effective in obtaining the desired effect. The device further includes a first coating layer. The first coating layer is permeable to the passage of the agent. In addition, the device includes a second coating layer which includes at least one impermeable disc and an impermeable polymer. The second coating layer is essentially impermeable to the passage of the agent and covers a portion of the first coating layer and inner core. The second coating layer blocks passage of the agent from the inner core at those sides where it contacts the first coating layer. The remaining portion of the inner core which is not blocked allows a controlled amount of the agent from the inner core to pass into the first coating layer via a passage in the second coating layer, into a third coating layer. The third coating layer is permeable to the passage of the agent and covers essentially the entire second coating layer. The second coating layer is positioned between the inner core and the third coating layer in order to control the rate at which the agent permeates through the third coating layer.

More specifically, the inventors discovered a device and method of preparation thereof that is suitable for the controlled and sustained release of an agent effective in obtaining a desired local or systemic physiological or pharmacological effect. In particular, it has been found that by sealing at least one surface with an impermeable disc, thinner coatings may be utilized. This has the advantage of enabling thinner, shorter devices to be prepared than otherwise possible. A further advantage is that as the material used to prepare the impermeable disc need not be malleable (to facilitate covering of a curved surface); instead relatively hard materials can be used to ease creation of uniform diffusion ports.

The device includes an inner core or reservoir which contains an agent effective in obtaining a desired effect. The device further includes a first coating layer, a second coating layer and a third coating layer. The first coating layer which is permeable to the passage of the effective agent may completely cover the inner core. The second coating layer covers only a portion of the first coating layer and inner core and is impermeable to the passage of the agent. The third coating layer covers all of the first coating layer and second coating layer and is permeable to the passage of the agent. The portion of the first coating layer and inner core that is not coated with the second coating layer facilitates passage of the agent through the third coating layer. Specifically, the second coating layer is positioned between the inner core and the third coating layer such that it blocks the passage of the agent through the adjacent portions of the third coating layer thus controlling the rate of passage of the agent.

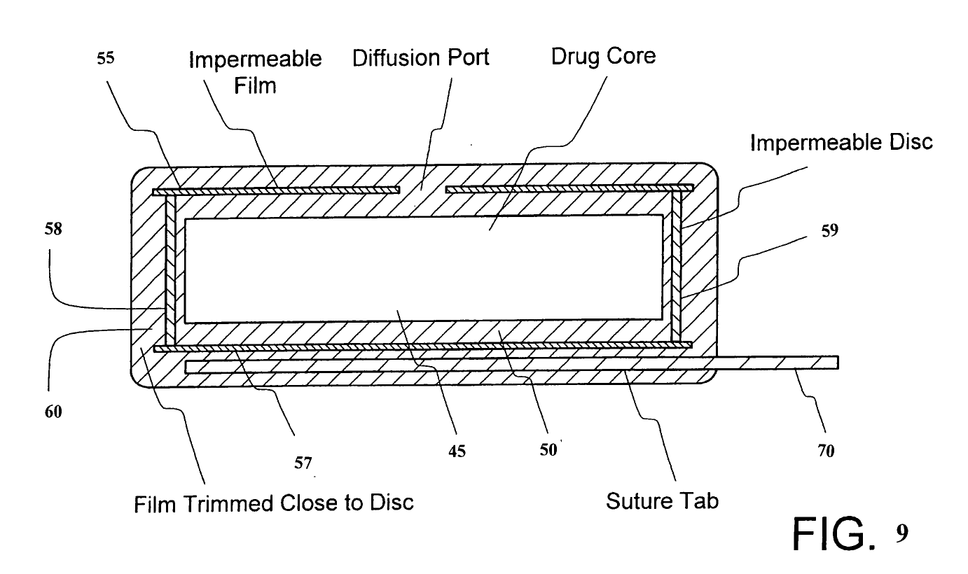

FIG. 9 illustrates one embodiment of the sustained release drug delivery device of the present invention. While the device shown in FIG. 9 is cylindrical, the device could be any shape. The device comprises an inner core or reservoir 45, a permeable coating 50 which is permeable to the passage of the agent in the core or reservoir, an impermeable coating 55 which is impermeable to the passage of the agent in the core or reservoir 45, and a permeable coating 60 which is permeable to the passage of the agent in the core or reservoir 45. The second coating includes an impermeable polymer 57 and discs 58 and 59 at the ends of the cylindrical core. FIG. 9 further shows a suture tag 70.

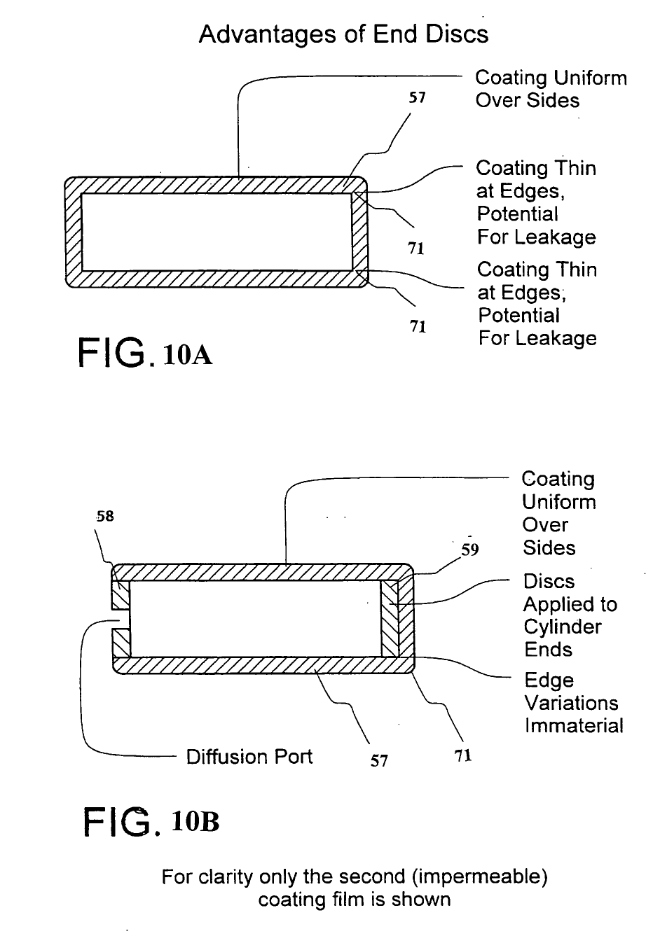

FIGS. 10A and 10B show only the second coating layer and illustrate the benefits associated with using impermeable discs as a portion of the second layer. FIG. 10A shows the impermeable polymeric layer 57 thinly coating the edges of the inner core. The thinly coated edges 71 create a potential for leakage of the effective agent.

FIG. 10B illustrates the benefits of using impermeable discs. The second coating layer contains the impermeable polymer 57 and the impermeable discs 58 and 59 at the ends of the cylindrical core. The impermeable disc 58 contains a diffusion port. The impermeable discs 58 and 59 prevent the leakage of the effective agent through the thin edges 71 of the impermeable polymer.

The devices are particularly suitable for treating ocular conditions such as glaucoma, proliferative vitreoretinopathy, diabetic retinopathy, uveitis, and keratitis. The devices are also particularly suitable for use as an ocular device in treating mammalian organisms suffering from cytomegalovirus retinitis wherein the device is surgically implanted within the vitreous of the eye.

A large number of polymers can be used to construct the devices of the present invention. The only requirements are that they are inert, non-immunogenic and of the desired permeability.

Materials that may be suitable for fabricating the device include naturally occurring or synthetic materials that are biologically compatible with body fluids and eye tissues, and essentially insoluble in body fluids with which the material will come in contact. The use of rapidly dissolving materials or materials highly soluble in eye fluids are to be avoided since dissolution of the wall would affect the constancy of the drug release, as well as the capability of the system to remain in place for a prolonged period of time.

Naturally occurring or synthetic materials that are biologically compatible with body fluids and eye tissues and essentially insoluble in body fluids which the material will come in contact include, but are not limited to, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale copolymer, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

Specifically, the second layer of the device of the present invention may be made of any of the above-listed polymers or any other polymer which is biologically compatible with body fluids and eye tissues, essentially insoluble in body fluids which the material will come in contact and essentially impermeable to the passage of the effective agent. The term impermeable, as used herein, means that the layer will not allow passage of the effective agent at a rate required to obtain the desired local or systemic physiological or pharmacological effect.

The second layer must be selected to be impermeable, as described above, to the passage of the agent from the inner core out to adjacent portions of the second coating layer. The purpose is to block the passage of the agent to those portions and thus control the release of the agent out of the drug delivery device.

The composition of the second layer, e.g., the polymer, must be selected so as to allow the above-described controlled release. The preferred composition of the second layer will vary depending on such factors as the active agent, the desired rate of control and the mode of administration. The identity of the active agent is important since the size of the molecule, for instance, is critical in determining the rate of release of the agent into the second layer.

The disc is essentially impermeable to the passage of the effective agent and may cover a portion of the inner core not covered by the impermeable film of the second coating layer. As shown in FIG. 10B, the disc may cover edges of the inner core and enables a thinner uniform coat of the impermeable film to be applied over the inner core than would otherwise be possible. In one embodiment, the impermeable film may completely cover the inner core and the discs. Drug release may occur via passage through a hole in the disc (see FIG. 10B) or a hole in the impermeable film. The physical properties of the polymer used for the disc can be selected based on their ability to withstand subsequent processing steps (such as heat curing) without suffering deformation of the hole. The polymer for the impermeable film can be selected based on the ease of coating the inner core. Possible materials for the disc include, Teflon, polycarbonate, polymethyl methacrylate, polyethylene alcohol, high grades of ethylene vinyl acetate (9% vinyl, content) and polyvinyl alcohol.

Since the second coating layer is essentially impermeable to the passage of the effective agent, only a portion of the inner core or reservoir and first coating layer may be coated with the second coating layer. Depending on the desired delivery rate of the device, the second coating layer may coat only a small portion of the surface area of the inner core for faster release rates of the effective agent or may coat large portions of the surface area of the inner core for slower release rates of the effective agent.

At least 50% of the surface area may be coated by the second coating layer. For slower release rates, at least 75% of the surface area may be coated. For even slower release rates, at least 95% of the surface area may be coated.

Thus, any portion of the surface area of the first coating layer and inner core up to but not including 100% may be coated with the second coating layer as long as the desired rate of release of the agent is obtained.

The second coating, including the impermeable film and impermeable disc, may be positioned anywhere over the inner core and first coating layer, including but not limited to the top, bottom or any side of the first coating layer and inner core. In addition, it could be on the top and a side, or the bottom and a side, or the top and the bottom, or on opposite sides or on any combination of the top, bottom or sides.

The first and third layer of the device of the present invention must be biologically compatible with body fluids and eye tissues, essentially insoluble in body fluids which the material will come in contact and permeable to the passage of the agent or composition effective in obtaining the desired effect.

The effective agent diffuses in the direction of lower chemical potential, i.e., toward the exterior surface of the device. At the exterior surface of the device, equilibrium is again established. When the conditions on both sides of the third coating layer are maintained constant, a steady state flux of the effective agent will be established in accordance with Fick's Law of Diffusion. The rate of passage of the drug through the material by diffusion is generally dependent on the solubility of the drug therein, as well as on the thickness of the wall. This means that selection of appropriate materials for fabricating the wall will be dependent on the particular drug to be used.

The rate of diffusion of the effective agent through a polymeric layer of the present invention may be determined via diffusion cell studies carried out under sink conditions. In diffusion cell studies carried out under sink conditions, the concentration of drug in the receptor compartment is essentially zero when compared to the high concentration in the donor compartment. Under these conditions, the rate of drug release is given by:

Q/t=(D·K·A·DC)/h

where Q is the amount of drug released, t is time, D is the diffusion coefficient, K is the partition coefficient, A is the surface area, DC is the difference in concentration of the drug across the membrane, and h is the thickness of the membrane.

In the case where the agent diffuses through the layer via water filled pores, there is no partitioning phenomena. Thus, K can be eliminated from the equation. Under sink conditions, if release from the donor side is very slow, the value DC is essentially constant and equal to the concentration of the donor compartment. Release rate therefore becomes dependent on the surface area (A), thickness (h) and diffusivity (D) of the membrane. In the construction of the device of the present invention, the size (and therefore, surface area) is mainly dependent on the size of the effective agent.

Thus, permeability values may be obtained from the slopes of a Q versus time plot. The permeability P, can be related to the diffusion coefficient D, by:

P=(K·D)/h

Once the permeability is established for the coating permeable to the passage of the agent, the surface area of the agent that must be coated with the coating impermeable to the passage of the agent may be determined. This is done by progressively reducing the available surface area until the desired release rate is obtained.

Exemplary microporous materials suitable for use as a first and third coating layer, for instance, are described in U.S. Pat. No. 4,014,335 which is incorporated herein by reference in its entirety. These materials include cross-linked polyvinyl alcohol, polyolefins or polyvinyl chlorides or cross-linked gelatins; regenerated, insoluble, nonerodible cellulose, acylated cellulose, esterified celluloses, cellulose acetate propionate, cellulose acetate butyrate, cellulose acetate phthalate, cellulose acetate diethyl-aminoacetate; polyurethanes, polycarbonates, and microporous polymers formed by co-precipitation of a polycation and a polyanion modified insoluble collagen. Cross-linked polyvinyl alcohol is preferred. The third coating layer is selected so as to slow release of the agent from the inner core into contact with a mammalian organism, e.g., a human. The third coating layer need not provide gradual release or control of the agent into the biological environment, however, the third coating layer may be advantageously selected to also have that property or feature.

The devices of the invention may be made in a wide variety of ways, such as by obtaining an effective amount of the agent and compressing the agent to a desired shape. Once shaped, the first coating layer may be applied. The first coating layer may be applied by dipping the device one or more times in a solution containing the desired polymer. Optionally, the first coating may be applied by dropping, spraying, brushing or other means of coating the outer surface of the device with the polymer solution. When using a polyvinyl alcohol solution to obtain the second coating layer, the desired thickness may be obtained by applying several coats. Each coat may be dried prior to applying the next coat. Finally, the device may be heated to adjust the permeability of the outer coating.

The impermeable disc may be applied directly over the first layer before coating with the impermeable polymer layer. In the case of a cylindrical core, an impermeable film may be wrapped around the core after discs are applied to one or both ends. Thus, the second coating layer includes both the impermeable film and the impermeable discs. By sealing at least one surface with an impermeable disc, thinner layers may be utilized. This has the advantage of enabling thinner, shorter devices to be prepared than otherwise possible.

The impermeable polymer layer should be thick enough to prevent release of drug across it except for the area not covered (the diffusion layer or port). Due to the desirability of minimizing the size of the implants, the thickness of the impermeable film layer therefore can be 0.01 to 2 millimeters, preferably 0.01 to less than 0.5 millimeters.

The impermeable disc should also be thick enough to prevent drug release across it save though [sic] a specifically prepared membrane or port. Due to the desirability of minimizing the size of the implants, the thickness of the impermeable disc can be 0.01 to 2 millimeters, preferably 0.01 to less than 1 millimeter.

Once the second coating layer, including the impermeable disc(s), is applied to the device, the third coating layer may be applied. The third coating may be applied by dipping the device one or more times in a solution containing the desired polymer. Optionally, the third coating layer may be applied by dropping, spraying, brushing or other means of coating the outer surface of the device with the polymer solution. When using a polyvinyl alcohol solution to obtain the third coating layer, the desired thickness may be obtained by applying several coats. Each coat may be dried prior to applying the next coat. Finally, the device may be heated to adjust the permeability of the outer coating.

The above description of how to make the devices of the present invention is merely illustrative and should not be considered as limiting the scope of the invention in any way, as various compositions are well known by those skilled in the art. In particular, the methods of making the device depends on the identity of the active agent and polymers selected. Given the active agent, the composition of the first coating, the second coating (the film and disc), and the third coating, one skilled in the art could easily make the devices of the present invention using conventional coating techniques.

The drug delivery system of the invention may be administered to a mammalian organism via any route of administration known in the art. Such routes of administration include intraocular, oral, subcutaneous, intramuscular, intraperitoneal, intranasal, dermal, and the like. In addition, one or more of the devices may be administered at one time or more than one agent may be included in the inner core.

The drug delivery system of the invention is particularly suitable for direct implantation into the vitreous of the eye and for application to an intraocular lens.

These methods of administration and technique for their preparation are well known by those of ordinary skill in the art. Techniques for their preparation are set forth in Remington's Pharmaceutical Sciences.

The drug delivery system may be administered for a sufficient period of time and under conditions to allow treatment of the disease state of concern.

For localized drug delivery, the devices may be surgically implanted at or near the site of action. This is the case for devices of the present invention used in treating ocular conditions, primary tumors, rheumatic and arthritic conditions, and chronic pain.

For systemic relief, the devices may be implanted subcutaneously, intramuscularly or intraperitoneally. This is the case when devices are to give sustained systemic levels and avoid premature metabolism. In addition, such devices may be administered orally.

When such devices are prepared for implantation within the vitreous of the eye, it is preferred that the device does not exceed about 7 millimeters in any direction. Thus, the cylindrical device shown in FIG. 9 would preferably not exceed 7 millimeters in height or 3 millimeters in diameter. The preferred thickness of the first coating layer ranges from about 0.05 to about 0.5 millimeters. The preferred thickness of the second coating layer ranges from about 0.1 to about 1.0 millimeters. The preferred thickness of the third coating layer ranges from about 0.1 to about 2.0 millimeters.

In another embodiment of the invention, an ocular device containing fluocinolone acetonide as the effective agent may be prepared. As further shown in the Examples which follow, such devices may be used to provide sustained release of fluocinolone acetonide for several years. The preferred amount of fluocinolone acetonide used in these devices ranges from 2 to 15 mg. More preferably, such devices contain approximately 5 to 10 mg. These preferred ranges may provide sustained release of the fluocinolone acetonide for a period of 3 years. The overall diameter of the device is 2 millimeters and the length is 5 millimeters.

The preferred materials include polyvinyl alcohol as the first layer, one end of the cylindrical device covered by a disc of ethylene vinyl acetate (9%) and the other uncovered, ethylene vinyl acetate (19%) as the impermeable polymer layer covering the sides of the cylinder, and the end sealed with the disc, and a third layer, polyvinyl alcohol, covering the entire assembly. The preferred thickness of the first layer ranges from 0.05 to 0.2 millimeters. The thickness of the impermeable polymer layer may range from 0.05 to 0.15 millimeters and is preferably 0.75 millimeters. The preferred thickness for the disc ranges from 0.05 to 2 millimeters and the preferred thickness of the third layer ranges from 0.1 to 0.5 millimeters.

By “vitreous” of the eye, it is meant the vitreous or vitreal cavity of the eye. By “aqueous” of the eye, it is meant the aqueous humor of the eye.

In the present invention, a sustained release device is implanted into the eye such that it delivers corticosteroid to the posterior segment of the eye. In a preferred embodiment, the sustained release device is implanted intravitreally. However, the device may also be implanted in the choroidal space, sub-retinally, or in the sclera. These methods of administration and techniques for their preparation are well known by those of ordinary skill in the art. Methods of administration and techniques for their preparation are set forth in Remington's Pharmaceutical Sciences.

The aqueous corticosteroid concentration remains less than the vitreous corticosteroid concentration for substantially the lifetime of the sustained release device. Thus, during release of the corticosteroid, the aqueous corticosteroid concentration is about 0.002 μg/ml to about 0.01 μg/ml, such as from about 0.01 μg/ml to about 0.05 μg/ml. Preferably, the aqueous corticosteroid concentration is less than about 0.05 μg/ml.

In contrast, during release of the corticosteroid, the vitreous corticosteroid concentration remains therapeutic, that is, less than about 10 μg/ml. The exact desired concentration depends upon the disease and therapeutic index of the drug.

The sustained release device useful in the present invention is any device which can be implanted to deliver corticosteroid to the vitreous of the eye and can release a corticosteroid for a sustained period of time, that is, for about 1 month to about 20 years, such as from about 6 months to about 5 years.

The sustained release device can be prepared to release the corticosteroid by pseudo zero order kinetics with a mean release rate of about 1 μg/day to about 50 μg/day, such as, about 1 μg/day to about 10 μg/day.

The following non-limiting examples are given by way of illustration only.

### EXAMPLE 1

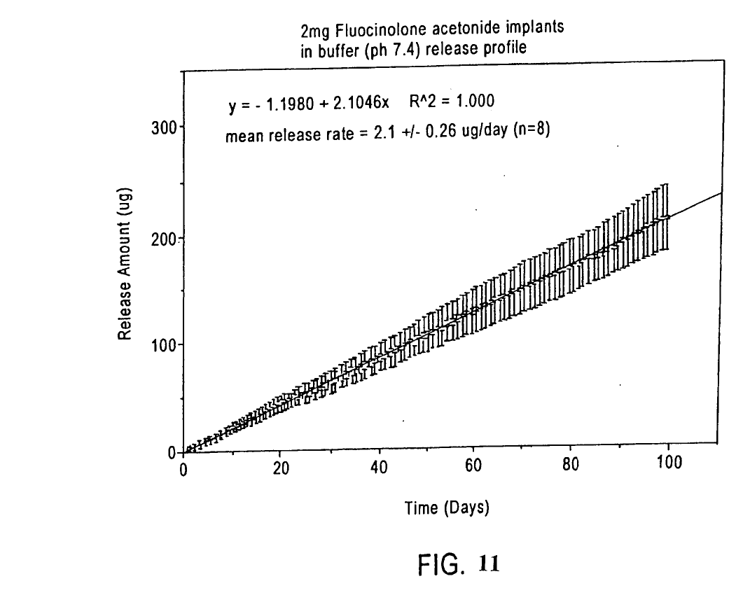

Sustained release fluocinolone acetonide devices were implanted into the vitreous of 4 rabbits while animals in the control group received a sham operation. After implantation, all rabbits received a sub-retinal injection of gelatin microspheres releasing basic fibroblast growth factor. All control animals developed neovascularization while ¾ of the implant group showed no evidence of neovascularization. No animals showed any indication of ocular or systemic steroid-induced toxicity. See FIG. 11.

### EXAMPLE 2

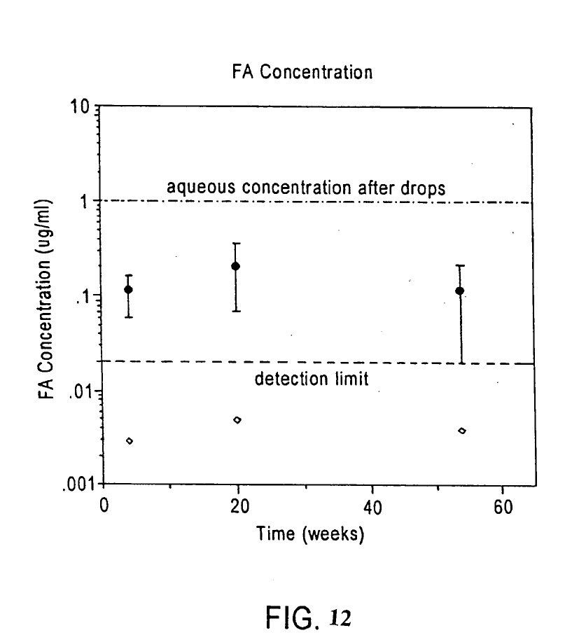

Animals received intravitreal fluocinolone acetonide implants and were sacrificed at 1 month, 4 months, and 11 months. Samples of the vitreous and aqueous were collected for analysis by HPLC. Analysis was performed using a fully automated Hitachi HPLC system. The mobile phase was 40% acetonitrile buffered to a pH of 4.0. The flow rate was 1.0 ml/min with an Axxion C-18 column (25 cm×4 mm×5 μm) and UV detection at 238 nm. Intravitreal levels were found to be relatively constant throughout the study (0.1-0.2 μg/ml) while no steroid was detected in the aqueous humor (limit of detection 0.02 μg/ml). See FIG. 12.

In the previous descriptions, numerous specific details are set forth, such as specific materials, structures, chemicals, processes, etc., in order to provide a better understanding of the present invention. However, the present invention can be practiced without resorting to the details specifically set forth. In other instances, well-known processing structures have not been described in detail in order not to unnecessarily obscure the present invention.

Only the preferred embodiment of the invention and but a few examples of its versatility are shown and described in the present disclosure. It is to be understood that the present invention is capable of use in various other combinations and environments and is capable of changes or modifications within the scope of the inventive concept as expressed herein. All patents, patent applications and publication cited herein are incorporated by reference in their entirety.

We claim:

1. A method for administering a corticosteroid to a patient, comprising implanting a sustained release drug device in a posterior segment of an eye of said patient, said drug delivery device comprising;
(i) an inner core including one or more corticosteroids to be released;
(ii) an impermeable coating which is impermeable to the passage of said corticosteroid(s) and including one or more pores through which said corticosteroid(s) diffuse, said impermeable coating being essentially insoluble and inert in body fluids, and compatible with eye tissues; and
(iii) one or more permeable coatings which are permeable to the passage of said corticosteroid(s), said permeable coatings being essentially insoluble and inert in body fluids, and compatible with eye tissues;
wherein said impermeable and permeable coatings are disposed about said inner core so as to produce, when implanted, a constant release of said corticosteroid(s) from said device at a rate that is not dependent on the rate of dissolution of the inner core, and which does not result in toxic amounts of the corticosteroid(s) accumulating in the aqueous humor of the eye.

2. The method of claim 1, wherein said device is implanted intravitreally.

3. The method of claim 2, wherein the vitreous corticosteroid concentration is less than 10 μg/ml.

4. The method of claim 1, wherein during release of the corticosteroid(s), the aqueous humor corticosteroid concentration is non-toxic.

5. The method of claim 4, wherein the aqueous humor corticosteroid concentration is less than 0.05 μg/ml.

6. The method of claim 1, as part of treatment or prevention for a disease state selected from the group consisting of ocular neovascularization, ocular inflammation, retinal degeneration, and retinopathy.

7. The method of claim 1, for delivering said corticosteroid(s) to the retina, retinal pigment epithelium (RPE) or choroids of the eye.

8. The method of claim 1, wherein said corticosteroid is selected from triamcinolone, dexamethasone, cortisone, prednisolone, flumetholone, and derivatives thereof.

9. The method of claim 1, wherein said corticosteroid is fluocinolone.

10. The method of claim 1, wherein said corticosteroid is fluocinolone acetonide.

11. The method of claim 1, wherein said corticosteroid is loteprednol.

12. The method of claim 4, wherein said corticosteroid is fluocinolone.

13. The method of claim 4, wherein said corticosteroid is fluocinolone acetonide.

14. The method of claim 4, wherein said corticosteroid is loteprednol.

15. The method of claim 6, wherein said corticosteroid is fluocinolone.

16. The method of claim 6, wherein said corticosteroid is fluocinolone acetonide.

17. The method of claim 6, wherein said corticosteroid is loteprednol.

18. The method of claim 1, wherein release of said corticosteroid(s) from said device follows zero order kinetics for at least 100 days.

19. The method of claim 1, wherein said sustained release device has a mean release rate for said corticosteroid(s) of 1 μg/day to 50 μg/day.

20. The method of claim 14, wherein said sustained release device has a mean release rate for said corticosteroid(s) of 1 μg/day to 10 μg/day.

21. The method of claim 1, wherein said constant release of said corticosteroid(s) includes a linear release of said corticosteroid(s) for at least 100 days.

22. The method of claim 1, wherein said device releases said corticosteroid(s) for 1 month to about 20 years.

23. The method of claim 1, wherein said device releases said corticosteroid(s) for 6 months to 5 years.

24. The method of claim 1, wherein said permeable coating(s) comprises a polymer coating selected from polyvinyl alcohols; polyolefins; polyvinyl chlorides; cross-linked gelatins; insoluble, non-erodible cellulose; polyurethanes; polycarbonates; and microporous polymers formed by co-precipitation of a polycation and a polyanion modified insoluble collagen.

25. The method of claim 1, wherein said permeable coating(s) comprises polyvinyl alcohol.

26. The method of claim 1, wherein said impermeable coating comprises ethylene vinyl acetate.

27. The method of claim 1, wherein said impermeable coating comprises polyvinyl acetate.

28. The method of claim 1, wherein said impermeable coating comprises polyimide.

29. The method of claim 1, wherein said impermeable coating comprises a silicone polymer.

30. The method of claim 1, wherein said pores in said impermeable coating of said device are less than 25% of the surface area.

31. The method of claim 1, wherein said pores in said impermeable coating are less than 5% of the surface area.

32. The method of claim 1, wherein said pores in said impermeable coating are less than 1% of the surface area.

33. The method of claim 1, wherein said sustained release device is attached to an intraocular lens or the haptic extending from said intraocular lens.

34. The method of claim 1, as part of treatment or prevention for a disease state selected from the group consisting of: diabetic retinopathy; chronic glaucoma; retinal detachment; sickle cell retinopathy; senile macular degeneration; retinal neovascularization; subretinal neovascularization; rubeosis iritis inflammatory diseases; chronic posterior and pan uveitis; neoplasms; retinoblastoma; pseudoglioma; neovascular glaucoma; neovascularization resulting following a combined vitrectomy and lensectomy; vascular diseases retinal ischemia; choroidal vascular insufficiency; choroidal thrombosis; neovascularization of the optic nerve; diabetic macular edema; cystoid macular edema; macular edema; retinitis pigmentosa; retinal vein occlusion; proliferative vitreoretinopathy; angioid streak; and retinal artery occlusion.

35. An implantable, sustained release device for administering a corticosteroid to a posterior segment of an eye, said device comprising:
(i) an inner core including one or more corticosteroids to be released;
(ii) an impermeable coating which is impermeable to the passage of said corticosteroid(s) and including one or more pores through which said corticosteroid(s) diffuse, said impermeable coating being essentially insoluble and inert in body fluids, and compatible with eye tissues; and
(iii) one or more permeable coatings which are permeable to the passage of said corticosteroid(s), said permeable coatings being essentially insoluble and inert in body fluids, and compatible with eye tissues;
wherein said impermeable and permeable coatings are disposed about said inner core so as to produce, when implanted, a constant release of said corticosteroid(s) from said device at a rate that is not dependent on the rate of dissolution of the inner core, and which does not result in toxic amounts of the corticosteroid(s) accumulating in the aqueous humor of the eye.

36. The device of claim 35, dimensioned for intravitreal implantation.

37. The device of claim 35, wherein the rate of release of said corticosteroid(s) produces a vitreous corticosteroid concentration of less than 10 μg/ml.

38. The device of claim 35, wherein the rate of release of said corticosteroid(s) produces an aqueous humor corticosteroid concentration that is non-toxic.

39. The device of claim 38, wherein the aqueous humor corticosteroid concentration is less than 0.05 μg/ml.

40. The device of claim 35, for use as in treatment or prevention for a disease state selected from ocular neovascularization, ocular inflammation, retinal degeneration, and retinopathy.

41. The device of claim 35, for delivering said corticosteroid(s) to the retina, retinal pigment epithelium (RPE) or choroids of the eye.

42. The device of claim 35, wherein said corticosteroid is selected from triamcinolone, dexamethasone, cortisone, prednisolone, flumetholone, and derivatives thereof.

43. The device of claim 35, wherein said corticosteroid is fluocinolone.

44. The device of claim 35, wherein said corticosteroid is fluocinolone acetonide.

45. The device of claim 35, wherein said corticosteroid is loteprednol.

46. The device of claim 38, wherein said corticosteroid is fluocinolone.

47. The device of claim 38, wherein said corticosteroid is fluocinolone acetonide.

48. The device of claim 38, wherein said corticosteroid is loteprednol.

49. The device of claim 40, wherein said corticosteroid is fluocinolone.

50. The device of claim 40, wherein said corticosteroid is fluocinolone acetonide.

51. The device of claim 40, wherein said corticosteroid is loteprednol.

52. The device of claim 35, wherein release of said corticosteroid(s) from said device follows zero order kinetics for at least 100 days.

53. The device of claim 35, having a mean release rate for said corticosteroid(s) of 1 μg/day to 50 μg/day.

54. The device of claim 35, having a mean release rate for said corticosteroid(s) of 1 μg/day to 10 μg/day.

55. The device of claim 35, wherein said constant release of said corticosteroid(s) includes a linear release of said corticosteroid(s) for at least 100 days.

56. The device of claim 35, wherein said device releases said corticosteroid(s) for 1 month to about 20 years.

57. The device of claim 35, wherein said device releases said corticosteroid(s) for 6 months to 5 years.

58. The device of claim 35, wherein said permeable coating(s) comprises a polymer coating selected from polyvinyl alcohols; polyolefins; polyvinyl chlorides; cross-linked gelatins; insoluble, non-erodible cellulose; polyurethanes; polycarbonates; and microporous polymers formed by co-precipitation of a polycation and a polyanion modified insoluble collagen.

59. The device of claim 35, wherein said permeable coating(s) comprises polyvinyl alcohol.

60. The device of claim 35, wherein said impermeable coating comprises ethylene vinyl acetate.

61. The device of claim 35, wherein said impermeable coating comprises polyvinyl acetate.

62. The device of claim 35, wherein said permeable coating(s) comprises polyimide.

63. The device of claim 35, wherein said impermeable coating comprises a silicone polymer.

64. The device of claim 35, wherein said pores in said impermeable coating of said device are less than 25% of the surface area.

65. The device of claim 35, wherein said pores in said impermeable coating are less than 5% of the surface area.

66. The device of claim 35, wherein said pores in said impermeable coating are less than 1% of the surface area.

67. The method of claim 1, wherein said device maintains therapeutic concentration of said corticosteroid(s) in the eye for 1 month to about 20 years.

68. The method of claim 1, wherein said device maintains therapeutic concentration of said corticosteroid(s) in the eye for 6 months to 5 years.

69. The device of claim 35, wherein said device maintains therapeutic concentration of said corticosteroid(s) in the eye for 1 month to about 20 years.

70. The device of claim 35, wherein said device maintains therapeutic concentration of said corticosteroid(s) in the eye for 6 months to 5 years.
